(19) United States
(12) Patent Application Publication
(10) Pub. No.: US 2008/0097352 A1
(43) Pub. Date: Apr. 24, 2008

(54) Methods of fabricating microneedles with bio-sensory functionality

(76) Inventors: Patricia A. Beck, Palo Alto, CA (US); Ramesh Govinda Raju, Singapore (SG); Xio-An Sean Zhang, Palo Alto, CA (US)

Correspondence Address: HEWLETT PACKARD COMPANY, P O BOX 272400, 3404 E. HARMONY ROAD, INTELLECTUAL PROPERTY ADMINISTRATION, FORT COLLINS, CO, 80527-2400, US

(21) Appl. No.: 11/520,526

(22) Filed: Sep. 12, 2006

(51) Int. Cl. A61M 5/32

(52) U.S. Cl. 604/272; 29/874

(57) ABSTRACT

A method of fabricating a microneedle is disclosed. The method includes forming at least one recess in a substrate, the at least one recess comprising an apex, forming an electrically seed layer on the substrate including the at least one recess, forming at least one electrically nonconductive pattern on a portion of the seed layer, the at least one nonconductive pattern being a pattern for a sensory area, plating an electrically conductive material on the seed layer to create a plated layer with an opening that exposes a portion of the nonconductive pattern and separating the plated layer from the seed layer and the at least one nonconductive pattern to release a hollow microneedle comprising a tip and at least one sensory area.

### FIELD OF THE INVENTION

The invention is generally related to microneedles and more particularly to methods of fabricating microneedles with bio-sensory functionality.

### BACKGROUND OF THE INVENTION

In the medical field, hollow needles have been developed for delivering drugs or withdrawal of bodily fluids across biological barriers, such as skin. Recently sharp hollow microneedles, with a penetration depth of 50 μm to 4 mm have been developed. Such needles with a penetration depth of about 50-150 μm are designed to penetrate the skin but avoid the nerves. An array of microneedles may be combined with an analyte measurement system to provide a minimally invasive fluid retrieval and analyte sensing system. In medical and other fields, solid microneedles, often with blunt tips, are desirable as probes to sense electrical signals or to apply stimulation electrical signals, and hollow microneedles are useful as means for dispensing small volume of materials.

Methods for fabricating microneedles from silicon have been proposed to draw and dispense small volumes of fluid. However, silicon is a brittle and its fractured material is irritating. A need exists for fabricating robust microneedles that can perform a variety of useful functions.

### BRIEF DESCRIPTION OF THE DRAWINGS

FIG. 1 is a flow chart illustrating a method for fabricating a microneedle in accordance with one embodiment of the present invention.

FIGS. 2A-2E show cross-sectional views illustrating the method steps of FIG. 1.

FIG. 3 is a flow chart illustrating a method for fabricating a microneedle in accordance with yet another embodiment of the present invention.

FIGS. 4A-4E show cross-sectional views illustrating the method steps of FIG. 3.

FIG. 5 is a flow chart illustrating a method for fabricating a microneedle in accordance with yet another embodiment of the present invention.

FIGS. 6A-6E show cross-sectional views illustrating the method steps of FIG. 5.

FIG. 7 is a flow chart illustrating a method for fabricating a microneedle with a slanted tip in accordance with yet another embodiment of the present invention.

FIGS. 8A-8E show cross-sectional views illustrating the method steps of FIG. 7.

FIG. 9 shows a concentric needle configuration in accordance with an embodiment of the present invention.

FIG. 10 shows an exemplary embodiment of a method of forming microneedles with micro/nano porous drug release channels in accordance with an embodiment of the present invention.

FIG. 11 shows a porous microneedle in accordance with an embodiment of the present invention.

### DETAILED DESCRIPTION

The present invention relates to methods of fabricating microneedles with bio-sensory functionality. The following description is presented to enable one of ordinary skill in the art to make and use the invention and is provided in the context of a patent application and its requirements. Various modifications to the embodiments and the generic principles and features described herein will be readily apparent to those skilled in the art. Thus, the present invention is not intended to be limited to the embodiment shown but is to be accorded the widest scope consistent with the principles and features described herein.

As shown in the drawings for purposes of illustration, methods of fabricating microneedles with bio-sensory functionality are disclosed. In an embodiment, microneedles are fabricated with sensory areas either on the external surface or internal to the microneedle and functionalized to become reactive to a desired chemical. In varying embodiments, these sensory areas can be made hydrophobic and/or hydrophilic in order to sense or detect the presence of predetermined agent and promote material movement or confinement. Throughout this application the term conductive refers to electrically conductive properties and the term nonconductive refers to electrically nonconductive properties.

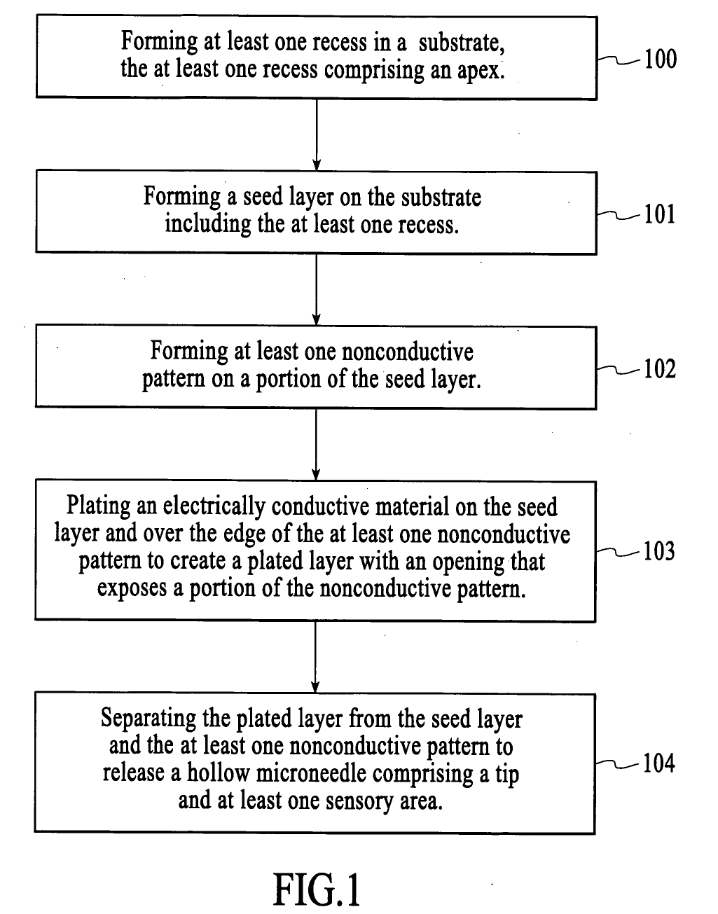

FIG. 1 is a flow chart illustrating a method for fabricating a microneedle in accordance with an embodiment. In this embodiment, a recess is formed in a substrate at step 100. In an embodiment, the recess includes an apex. An electrically conductive seed layer is formed on the substrate at step 101. An electrically nonconductive pattern is formed on a portion of the seed layer at step 102. At step 103, an electrically conductive material is plated on the seed layer and over the edge of the nonconductive pattern to create a plated layer with an opening that exposes a portion of the nonconductive pattern. Next, the plated layer is separated from the seed layer and the nonconductive pattern to release a hollow microneedle comprising a tip and at least one sensory channel at step 104.

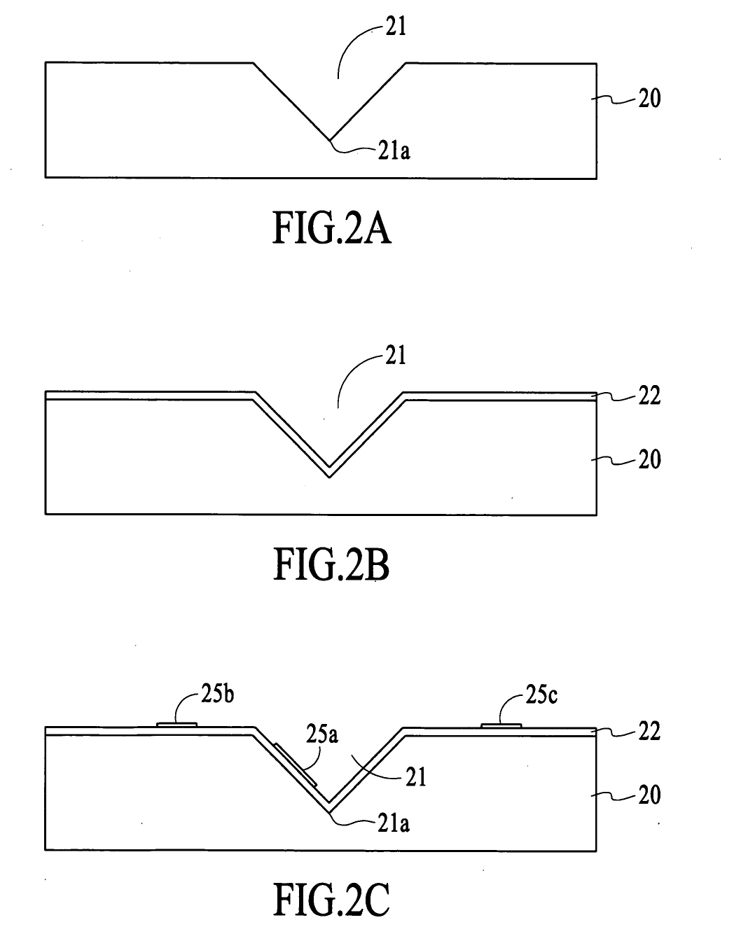

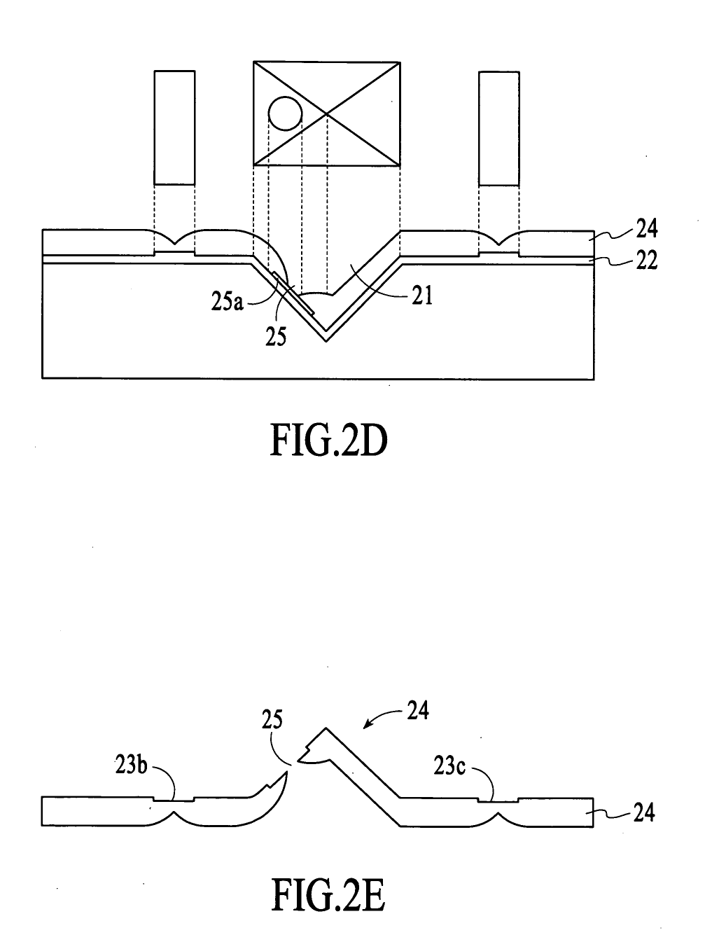

FIGS. 2A-2E show the cross-sectional views illustrating the method steps of FIG. 1. Referring to FIG. 2A, a recess 21 with an apex 21a is formed in a substrate 20. The substrate 20 can be constructed from a semiconductor material such as silicon, a dielectric, a nonconductive material such as glass, a metal such as stainless steel or aluminum, a premolded plastic or the like. In one embodiment, the substrate is made of silicon, and the recess is a pyramidal etch pit formed by masking the substrate and using a crystallographic etch

Referring to FIG. 2B, a seed layer 22 is formed over the surface of the substrate 20 such that the recess 21 is covered by the seed layer. The seed layer 22 may be a thin layer of chrome, stainless steel, tantalum or gold, or other conductive material which is formed by sputtering or other conventional deposition techniques. The seed layer 22 may also be a bilayer of chrome/stainless steel (the chrome layer being formed first in the sequence) or tantalum/gold (the tantalum layer being formed first in the sequence). The thickness for the seed layer may be between about 500 angstroms to about 200,000 angstroms.

Next, at least one nonconductive pattern 23a, 23b, 23c is formed over a portion of the seed layer 22 that is on a sidewall of the recess 21 as shown in FIG. 2C. A first nonconductive pattern 23a is in the recess 21 and laterally offset from the apex 21a as illustrated by the top view X in FIG. 2C. Second and third nonconductive patterns 23b, 23c are formed on opposite sides of the microneedle. The patterning of the nonconductive layer may be done by forming a photolithographic mask on the nonconductive layer followed by etching. Some suitable materials for the nonconductive pattern include silicon carbide (SiC), photoresist, other polymers, silicon nitride, silicon oxide. The thickness for the nonconductive pattern may be between about 500 angstroms to about 500,000 angstroms.

Referring to FIG. 2D, an electrically conductive material is electroplated onto the seed layer 22 and over the edge of the nonconductive pattern 23a to create a plated layer 24 with an offset opening 25 that exposes a portion of the nonconductive pattern 23a. The electrically conductive material used for forming the plated layer 24 is different from the electrically conductive material forming the seed layer 22. The microneedle may be constructed of a variety of metals or other conductive materials depending on the intended use. For medical applications, the microneedle may be made of palladium, silver, gold, nickel, brass, bronze, or alloys thereof. The microneedle may be made of one material coated with another, such as stainless steel coated with silver for antimicrobial properties, or a polymer coated with a metal or a metal coated with a polymer to yield various electrical, thermal, reactionary or other properties.

The plated layer 24 conforms to the shape of the recess 21 as shown in FIG. 2D to define the body of the microneedle. The opening 25 is a tapered through hole extending through the thickness of the microneedle. The location and shape of the nonconductive pattern 23a defines the location and shape of the opening 25. Referring to FIG. 2E, the plated layer 24 is separated from the seed layer 22 and the nonconductive pattern 23a to release a free-standing microneedle 24. FIG. 2F shows an isometric view of the microneedle 24 with the offset opening 25 and first and second sensory areas 23b and 23c.

The sensory areas 23a and 23b can be functionalized to sense and/detect a desired agent(s). These areas can be made chemically reactive in a hydrophobic or hydrophilic fashion to direct fluid flow along the inner and/or outer surfaces of the microneedle. The surface area of these sensory regions can be increased prior to being made chemically reactive in order to increase the reaction area. Increased surface area provides more area for detection of a chemical, reaction with a chemical and fosters increased sensitivity. Additionally, surrounding or adjacent material to the sensory area can also be made hydrophobic/hydrophilic etc. For example, one might make the inner surface of the microneedle hydrophobic except for the sensory area or the sensory area and wicking channels to the sensory areas.

The sensor may be a coating which has its resistance altered in response to a particular chemical. Taking, for example, the situation of needle extracting a sample and seeking a protein. The needle may have already injected a chemical to facilitate the collection of the protein, then without removing the needle from its inserted position fluid is drawn back up into the needle. If sensory areas 23a and 23b are formed which are preferentially reactive to a protein, then the resistance of the coated surface would change thereby sensing protein.

Although, the above-described embodiment is disclosed in a narrow context, one of ordinary skill will readily recognize that a variety of different implementations could by utilized while remaining within the spirit and scope of the present inventive concepts. For example, the outside of the needle could be used to sense whether or not a fluid is flowing from a fluidicly connected reservoir by using a sensor at the tip to detect the proper outflow of a chemical. If sensory areas are placed on the base of the microneedle, the sensory areas could be used to detect if fluid is coming back out around the microneedle rather than flowing through the orifice into the target. For example, hemoglobin sensors on the outside/inside of the microneedle, the presence of blood can be detected.

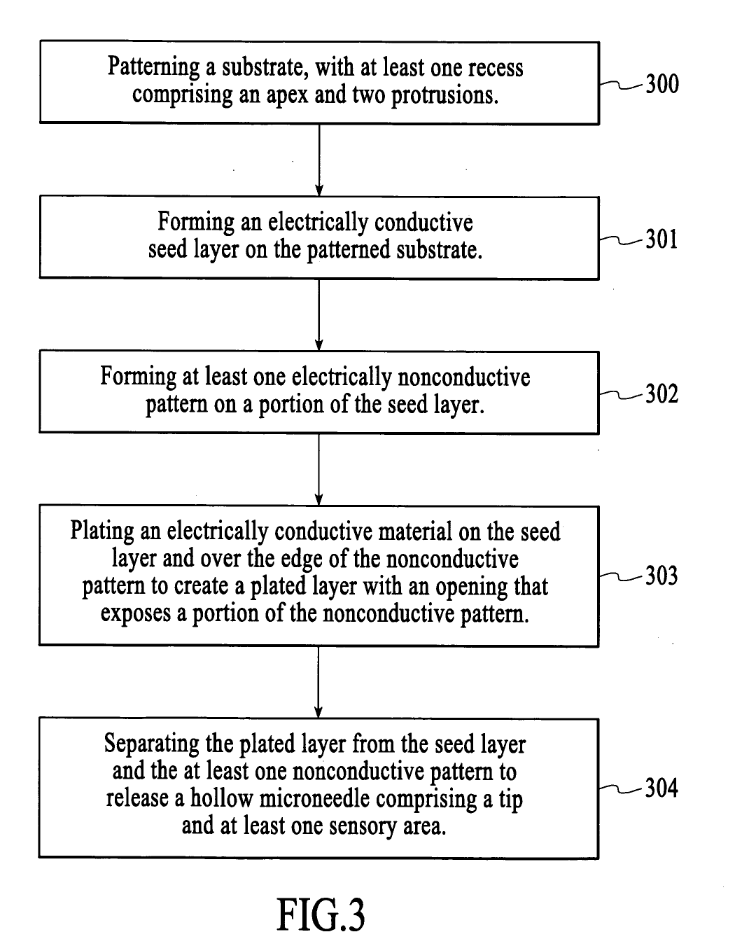

FIG. 3 is a flow chart illustrating a method for fabricating a microneedle in accordance with an alternate embodiment. In this embodiment, a substrate is patterned with two protrusions and a recess at step 300. In an embodiment, the recess includes an apex. An electrically conductive seed layer is formed on the patterned substrate at step 301. An electrically nonconductive pattern is formed on a portion of the seed layer at step 302. At step 303, an electrically conductive material is plated on the seed layer and over the edge of the nonconductive pattern to create a plated layer with an opening that exposes a portion of the nonconductive pattern. Next, the plated layer is separated from the seed layer and the nonconductive pattern to release a hollow microneedle comprising a tip and at least one sensory area at step 304.

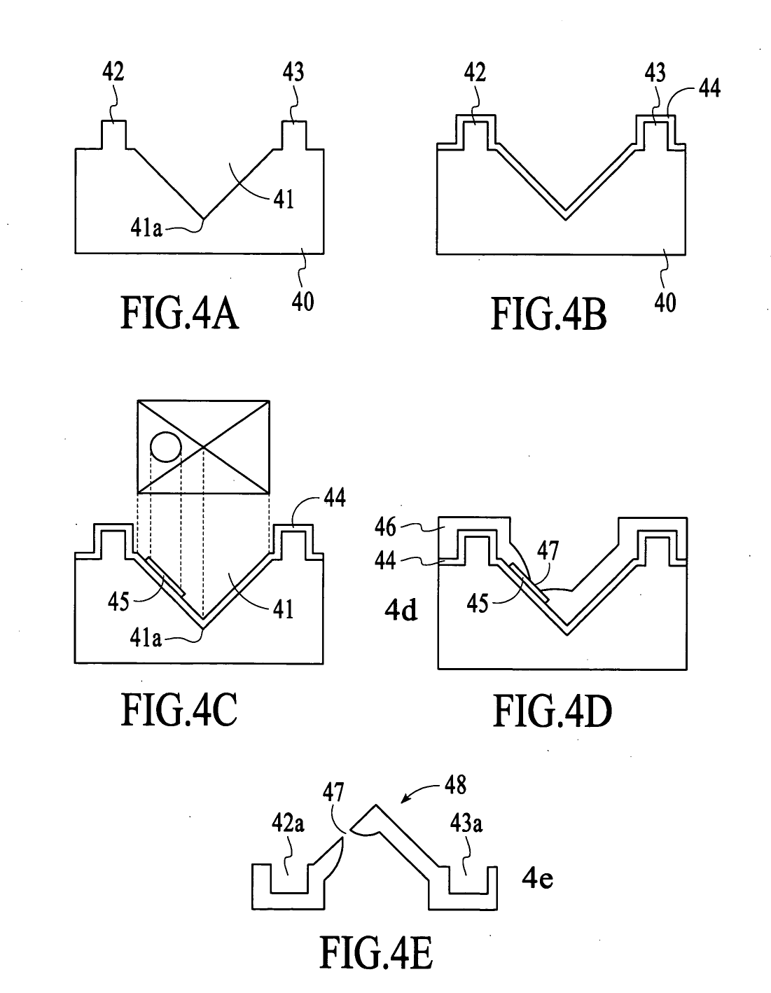

FIGS. 4A-4E show the cross-sectional views illustrating the method steps of FIG. 3. Referring to FIG. 4A, a substrate 40 is formed with a recess 41 with an apex 41a and protrusions 42, 43. The substrate 40 can be constructed from a semiconductor material such as silicon, a dielectric, a nonconductive material such as glass, a metal such as stainless steel or aluminum, or a premolded plastic. In one embodiment, the substrate is made of silicon, and the recess is a pyramidal etch pit formed by masking the substrate etching with a solution containing a crystallographic etch.

Referring to FIG. 4B, an electrically conductive seed layer 44 is formed over the top surface of the substrate 40 such that the recess 41 and the protrusions 42, 43 are covered by the seed layer 44. The seed layer 44 may be a thin layer of chrome, stainless steel, tantalum or gold, which is formed by sputtering or other conventional deposition techniques or other conductive material. The seed layer 44 may also be a bilayer of materials such as chrome/stainless steel the chrome layer being formed first or tantalum/gold (the tantalum layer being formed first). The thickness for the seed layer may be between about 500 angstroms to about 200,000 angstroms.

Next, at least one nonconductive pattern 45 is formed over a portion of the seed layer 44 that is on a sidewall of the recess 41 as shown in FIG. 4C. The nonconductive pattern 45b is in the recess 41 and laterally offset from the apex 41a. The patterning of the nonconductive layer may be done by forming a photolithographic mask on the nonconductive layer followed by etching, or by shadow masking or by lift-off lithography or directed deposition or by other suitable means. Some suitable materials for the nonconductive pattern 45 include silicon carbide (SiC), photoresist, other polymers, silicon nitride, silicon oxide. The thickness for the nonconductive pattern may be between about 500 angstroms to about 500,000 angstroms.

Referring to FIG. 4D, an electrically conductive material is electroplated onto the seed layer 44 and over the edge of the nonconductive pattern 45 to create a plated layer 46 with an offset opening 47 that exposes a portion of the nonconductive pattern 45. The electrically conductive material used for forming the plated layer 46 is different from the electrically conductive material forming the seed layer 44.

The plated layer 46 conforms to the shape of the recess 43 as shown in FIG. 4D to define the body of the microneedle. The opening 47 is a tapered through hole extending through the thickness of the microneedle. The location and shape of the nonconductive pattern 45 defines the location and shape of the opening 47. Referring to FIG. 4E, the plated layer 46 is separated from the seed layer 44 and the nonconductive pattern 45 to release a free-standing microneedle 48. FIG. 4E shows an isometric view of the microneedle 48 with the offset opening 47 and first and second sensory areas 42a and 43a.

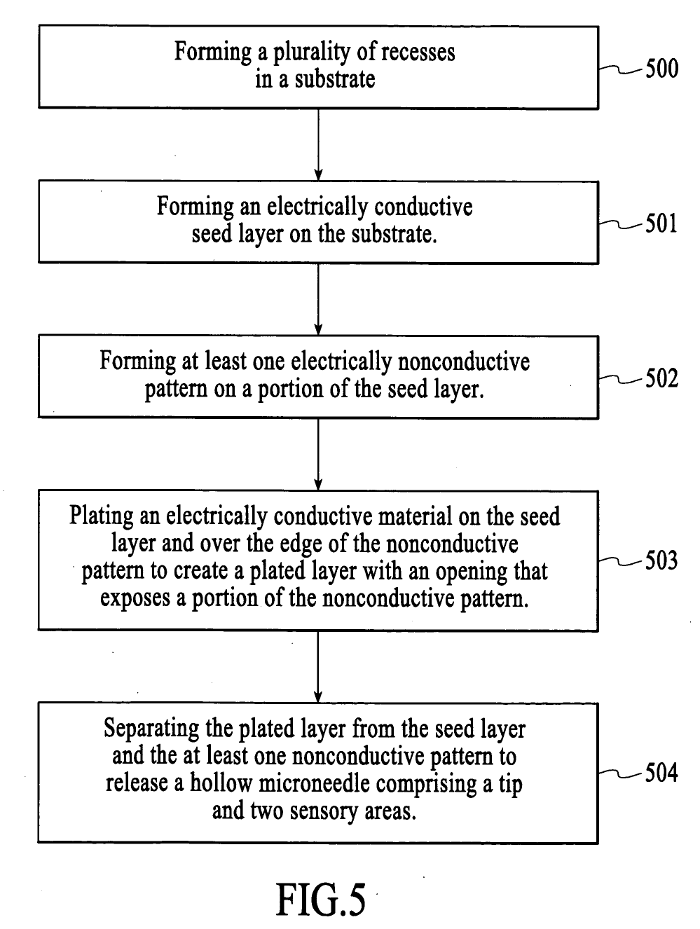

FIG. 5 is a flow chart illustrating a method for fabricating a microneedle in accordance with an embodiment. In this embodiment, a plurality of recesses are formed in a substrate at step 500. In an embodiment, each recess includes an apex. An electrically conductive seed layer is formed on the substrate at step 501. An electrically nonconductive pattern is formed on a portion of the seed layer at step 502. At step 503, an electrically conductive material is plated on the seed layer and over the edge of the nonconductive pattern to create a plated layer with an opening that exposes a portion of the non-conductive pattern. Next, the plated layer is separated from the seed layer and the nonconductive pattern to release a hollow microneedle comprising a tip and two sensory areas at step 504. In an embodiment, the seed layer is a different material than the subsequently plated layer.

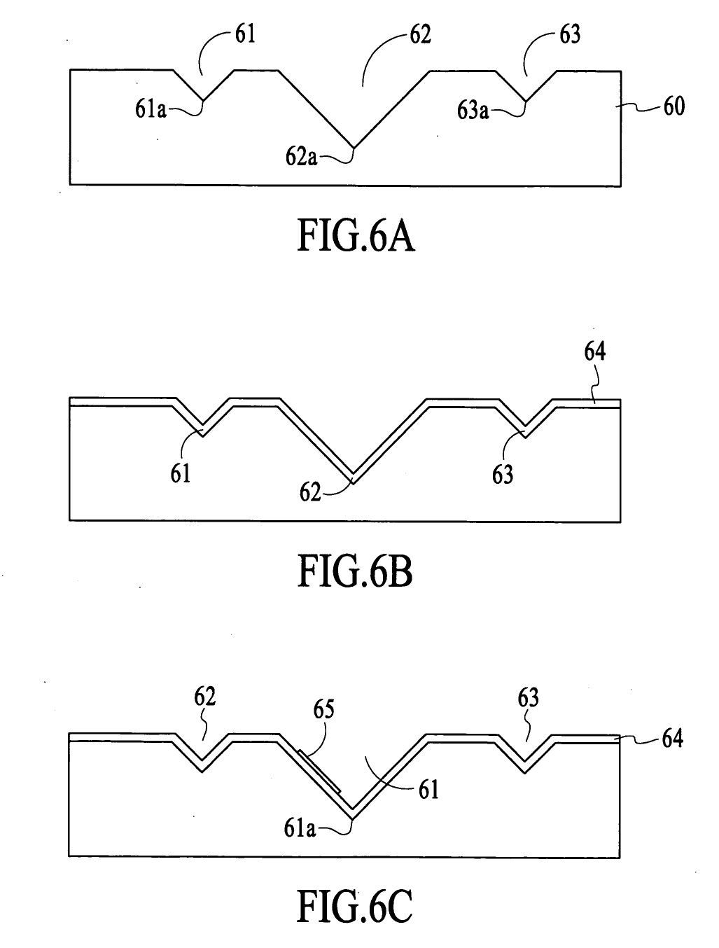

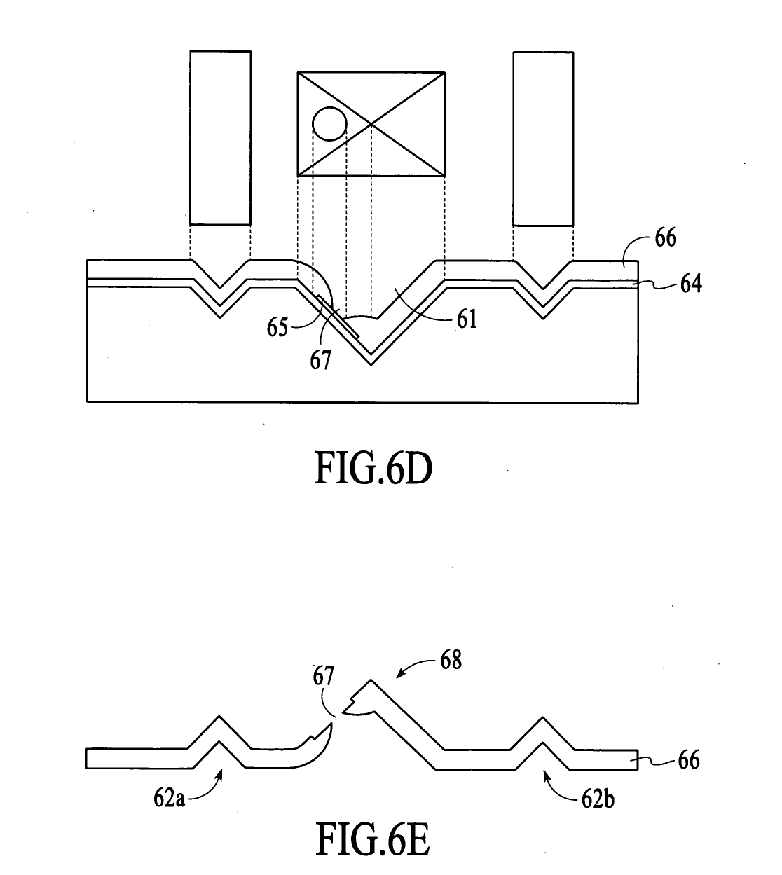

FIGS. 6A-6E show the cross-sectional views illustrating the method steps of FIG. 5. Referring to FIG. 6A, a substrate 60 is formed with three recesses 61, 62 and 63 with respective apexes 61a, 62a and 63a. Referring to FIG. 6B, a seed layer 64 is formed over the surface of the substrate 60 such that the recesses 61, 62 and 63 are covered by the seed layer 64. Again, the seed layer 64 may be an electrically conductive layer of chrome, stainless steel, tantalum or gold, which is formed by sputtering or other conventional deposition techniques. It should be understood that the seed layer may be formed of an electrically conductive material other than metal, e.g. conductive polymers. In addition, the materials forming the microneedle are not limited to metals but can also include electrically conductive materials other than metal, e.g. conductive polymers. In such case, the electrically conductive material forming the seed layer may be the same or different from the materials forming the microneedle.

Next, at least one nonconductive pattern 65 is formed over a portion of the seed layer 64 that is on the recesses 61, 62, 63 as shown in FIG. 6C. The nonconductive pattern 65 is in the recess 61 and laterally offset from the apex 61a. The patterning of the nonconductive layer may be accomplished by photolithographic methods such as lithographic masking, shadow masking, directed depositions.

Referring to FIG. 6D, an electrically conductive material is electroplated onto the seed layer 64 and over the edge of the nonconductive pattern 65 to create a plated layer 66 with an offset opening 67 that exposes a portion of the nonconductive pattern 65. In this embodiment the electrically conductive material used for forming the plated layer 66 is different from the electrically conductive material forming the seed layer 64.

The plated layer 66 conforms to the shape of the recess 61 as shown in FIG. 6D to define the body of the microneedle. The opening 67 is a tapered through hole extending through the thickness of the microneedle. The location and shape of the nonconductive pattern 65 defines the location and shape of the opening 67. Referring to FIG. 6E, the plated layer 66 is separated from the seed layer 64 and the nonconductive pattern 65 to release a free-standing microneedle 68. FIG. 6E shows an isometric view of the microneedle 68 with the offset opening 67 and first and second sensory areas 62a and 63a.

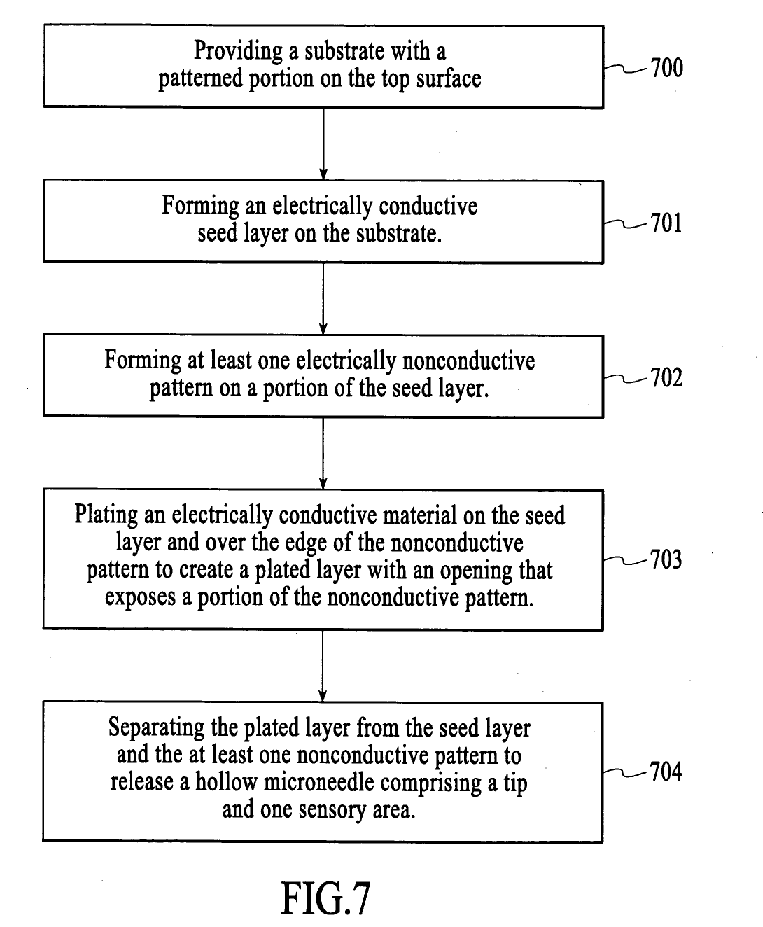

Although the above-described embodiments show the formation of sensory areas outside the microneedle itself, one of ordinary skill in the art will readily recognize that sensory areas can be formed inside the microneedle as well. FIG. 7 is a flow chart illustrating the processing sequence for fabricating sensory areas within the microneedle in accordance with another embodiment. In this embodiment, a substrate having a patterned portion on the top surface is provided at step 700. In an embodiment, the patterned portion is pyramidally shaped. An electrically conductive seed layer is formed on the top surface of the substrate at step 701. At least one electrically nonconductive pattern is formed on the seed layer at step 702 so that a portion of the nonconductive pattern is on the patterned portion. In an embodiment, a plurality of nonconductive patterns are formed on the patterned portion of the substrate.

At step 703, an electrically conductive material is plated on the seed layer and over the nonconductive patterns to create a plated layer with an opening that exposes a portion of the nonconductive patterns. Next, the plated layer is separated from the seed layer and the nonconductive patterns to release a hollow microneedle comprising a tip and at least one interior sensory channel at step 704.

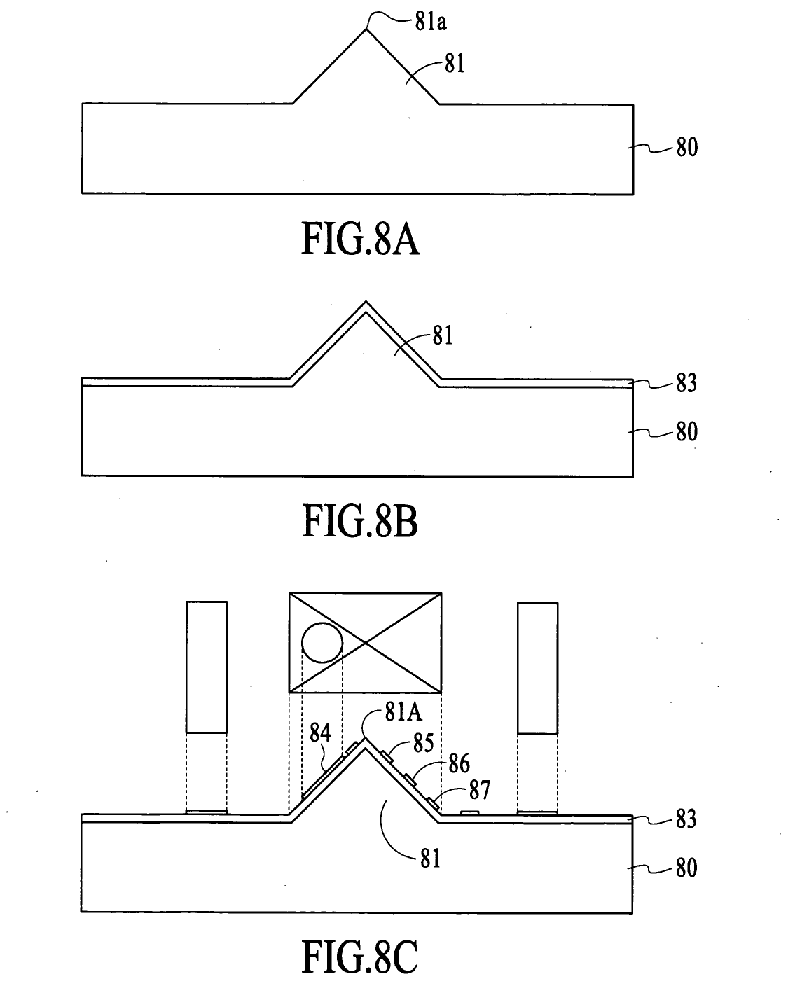

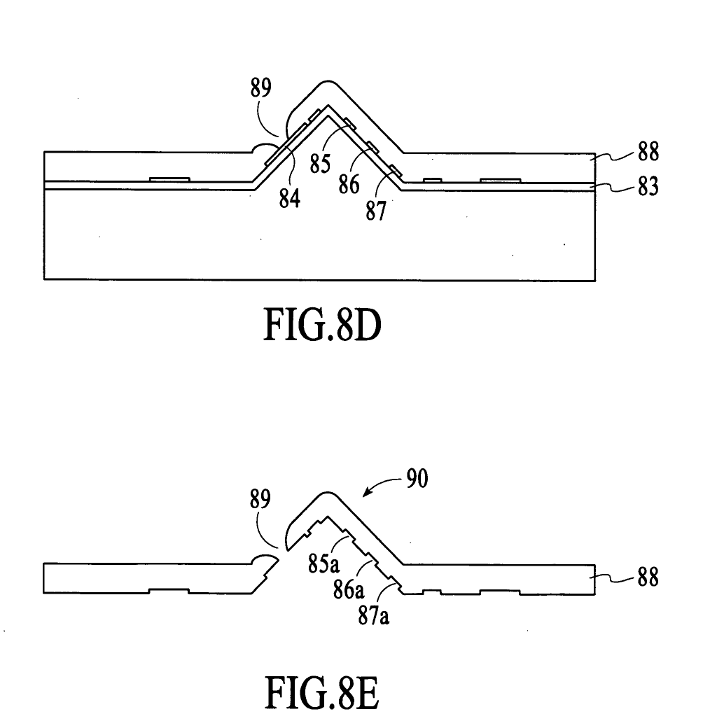

FIGS. 8A-8E show the cross-sectional views illustrating the method steps of FIG. 7. Referring to FIG. 8A, a substrate 80 is formed with a patterned portion 81 with an apex 81a. Referring to FIG. 8B, a seed layer 82 is formed over the surface of the substrate 80 such that the patterned portion 81 is covered by the seed layer 82. Again, the seed layer 82 may be a thin metal-containing layer of chrome, stainless steel, tantalum or gold, or other conductive material which is formed by sputtering or other conventional deposition techniques.

Next, the electrically nonconductive patterns 84, 85, 86 and 87 are formed over a portion of the seed layer 82 that is on the patterned portion 81 as shown in FIG. 8C. The nonconductive patterns 84, 85, 86 and 87 are near the apex 81a. The patterning of the nonconductive layer may be accomplished by photolithographic methods such as lithographic masking, shadow masking, directed depositions.

Referring to FIG. 8D, an electrically conductive material is electroplated onto the seed layer 82 and over the nonconductive patterns 84, 85, 86 and 87 to create a plated layer 88 with an offset opening 89 that exposes a portion of the nonconductive pattern 84. In this embodiment the electrically conductive material used for forming the plated layer 88 is different from the electrically conductive material forming the seed layer 82.

The plated layer 88 conforms to the shape of the apex 81a as shown in FIG. 8D to define the body of the microneedle. The opening 89 is a tapered through hole extending through the thickness of the microneedle. The location and shape of the nonconductive pattern 84 defines the location and shape of the opening 89. Referring to FIG. 8E, the plated layer 88 is separated from the seed layer 83 and the nonconductive patterns 84, 85, 86 and 87 to release a free-standing microneedle 90. FIG. 8E shows an isometric view of the microneedle 90 with the offset opening 89 and first, second and third interior sensory areas 85a, 86a and 87a. Interior sensory areas 85a, 86a and 87a could also be formed by etching or molding the substrate 80.

The microneedles fabricated by the above methods may have the following dimensions: a height in the range from about 2 μm to about 4 mm, a base diameter in the range from about 5 μm to about 1000 μm. For hollow microneedles, the luminal diameter (i.e., the diameter of the opening at the tip) is in the range from about 5 μm to about 150 μm.

All of the above methods can be adapted to co-fabricate an array of microneedles simultaneously. In such case, the method steps are the same as described above except that a plurality of microneedles are formed on a common substrate instead of just one. Other modifications to the above methods are also possible. For example, two or more different conductive materials may be used to form the plated microneedle shape (e.g., radially and/or vertically). The electroplating process can be controlled such that the tip of the microneedle is formed of a material different from the base of the microneedle. Furthermore, instead of plating metals onto a substrate to form the microneedle shape, conductive polymers may be plated. Although electroplating has been discussed in some embodiments, it should be understood by those skilled in the art that other conventional plating methods are possible.

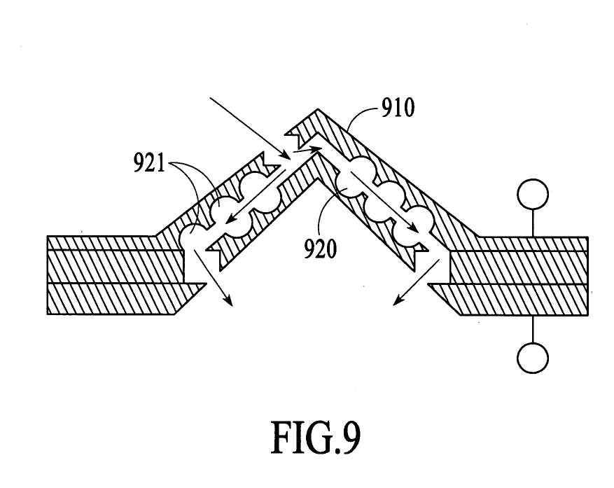

The microneedle fabricated by the above methods may be integrated with a measurement means to provide a fluid sampling and measurement device. For example, the sensor may constitute a patterned area in contact with or electrically isolate from the needle body or may constitute the entire inner or outer surface of the needle. For example, if employing concentric needles, the outer surface of one and the inner surface of another may serve as terminals to make the measurement. FIG. 9 shows a concentric needle configuration including an outer surface 910 and an inner surface 920 whereby the inner surface includes sensory areas 921. Furthermore, the hollow microneedle may be attached to a reservoir chamber that holds drugs, reagents, or other materials to be delivered for various applications, including therapeutic or diagnostic applications or is a collection reservoir.

Additionally, increasing the surface area of the above-described sensory areas may be accomplished by texturizing, intentionally shaping the surfaces, applying an etching agent (either wet or dry) to roughen the surfaces, applying a coating containing nanoparticulate matter or applying a Langmuir-Blodgett film of molecules which are expected to aggregate into domains and which will affect the plating of subsequent materials. This methodology can be employed to create microneedles with multiple micro or nano porous drug release channels.

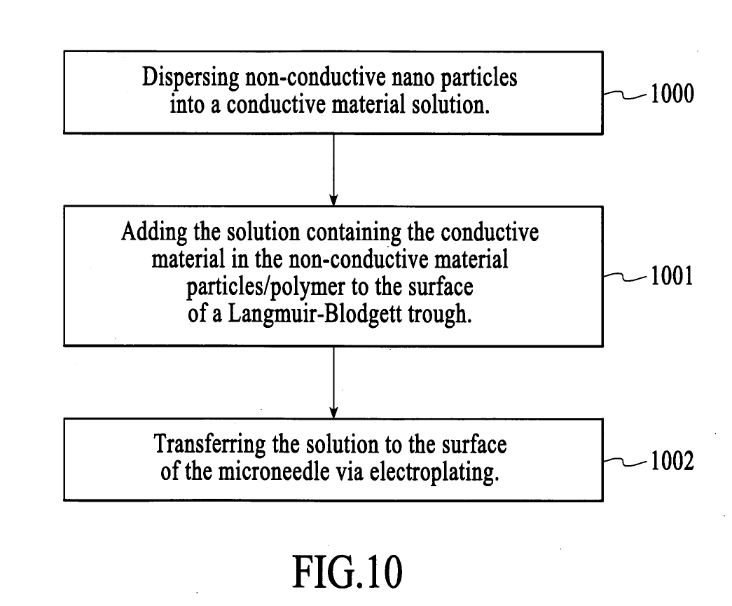

FIG. 10 shows an exemplary embodiment of a method of forming microneedles with micro/nano porous drug release channels. In this embodiment, non-conductive nano particles are dispersed into a conductive material solution, at step 1000. The ratio of non-conductive particles to conductive particles can be varied. The exact ratio depends upon the specific requirements (hole, size, thickness of material, viscosity, etc.). Next, the solution containing the conductive material and the non-conductive material particles/polymer is added to the surface of the Langmuir-Blodgett trough at step 1001. The amount of material added should be enough to form a monolayer of particles.

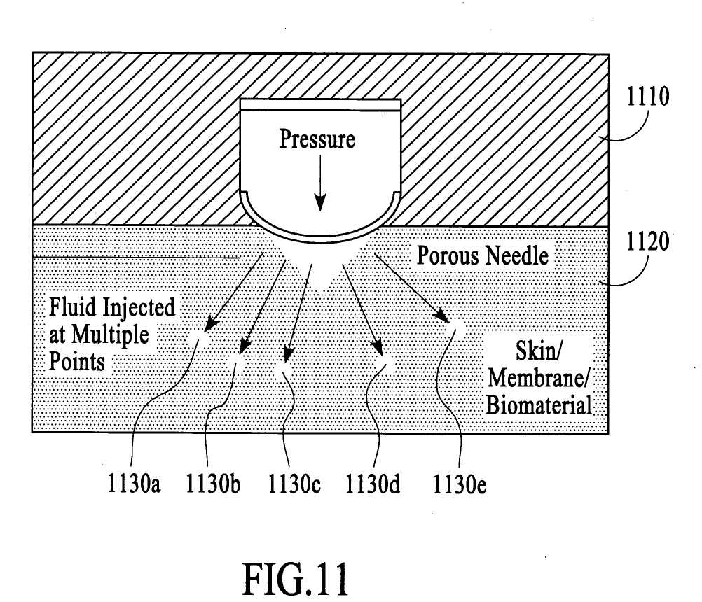

A next step 1002, includes transferring the solution to the surface of the microneedle. Subsequent electroplating will initiate in the regions of conductive materials. Plating coverage will be hindered in regions occupied by non-conductive particles thereby creating micro/nano porous drug releasing channels. Needles made in this fashion effectively distribute the drug/fluid of interest over a larger area, without multiple needle intrusion, for more effective assimilation and reactivity. FIG. 11 is an illustration of this concept. FIG. 11 shows a porous microneedle 1110 penetrating a membrane 1120 (skin, biomaterial, etc.) at multiple points 1130a, 1130b, 1130c, 1130d, 1130e. Accordingly, this type of implementation provides a patch-like diffusion in a subcutaneous or sub-membrane environment.

L-B techniques and Self-Assembled Monolayer techniques may also be used to functionalize the surfaces. With LB techniques, the surface may be uniformly coated with a reagent, if its hydrophilic properties are uniform. For site specific binding, such as only in the sensory areas, molecules with the desired properties may be preferentially bound to surfaces by use of a specific binding interface group. As an example, a thiol group may bind to Au or Pt but not to Tungsten. Once the area is covered in a fluid containing these binders, the agents self-assemble themselves only onto to the predetermined areas of interest.

As shown in the drawings for purposes of illustration, methods of fabricating microneedles with bio-sensory functionality are disclosed. In varying embodiments, microneedles are fabricated with sensory areas either on the external surface or internal to the microneedle and functionalized to become reactive to a desired agent thereby combining sensing with bioassay functionality. By combining sensing with bioassay functionality in the needle, an active probe is formed which has the capacity to withdraw fluids and analyze them immediately rather than transporting the sample to another chamber. Additionally, smaller sample volumes are required. Also, potential contamination is reduced or eliminated and accuracy is increased. Furthermore, fluids may be added to or withdrawn from the site allowing chemical reaction products to be checked without needle repositioning or reinsertion or the addition of other puncturing probes.

Without further analysis, the foregoing so fully reveals the gist of the present inventive concepts that others can, by applying current knowledge, readily adapt it for various applications without omitting features that, from the standpoint of prior art, fairly constitute essential characteristics of the generic or specific aspects of this invention. Therefore, such applications should and are intended to be comprehended within the meaning and range of equivalents of the following claims. Although this invention has been described in terms of certain embodiments, other embodiments that are apparent to those of ordinary skill in the art are also within the scope of this invention, as defined in the claims that follow.

1. A method of fabricating a microneedle, said method comprising:
(a) forming at least one recess in a substrate, the at least one recess comprising an apex;
(b) forming an electrically conductive seed layer on the substrate including the at least one recess;
(c) forming at least one electrically nonconductive pattern on a portion of the seed layer, the at least one nonconductive pattern being a pattern for a sensory area;
(d) plating an electrically conductive material on the seed layer to create a plated layer with an opening that exposes a portion of the nonconductive pattern; and
(e) separating the plated layer from the seed layer and the at least one nonconductive pattern to release a hollow microneedle comprising a tip and at least one sensory area.

2. The method of claim 1 wherein the sensory area comprises a channel.

3. The method of claim 1 wherein the at least one recess comprises a plurality of recesses, each of the plurality of recesses comprising an apex wherein the act of forming at least one nonconductive pattern on a portion of the seed layer further comprises:
forming at least one nonconductive pattern on a sidewall of one of the plurality of recesses.

4. The method of claim 1 wherein the substrate includes at least one patterned protrusion.

5. The method of claim 1 wherein another of the at least one nonconductive pattern is not on the recess.

6. The method of claim 3 wherein the act of forming at least one nonconductive pattern on a sidewall of one of the plurality of recesses further comprises:
forming at least one nonconductive pattern on both sidewalls of one of the plurality of recesses.

7. The method of claim 4 wherein the at least one sensory area comprises multiple sensory areas around the interior circumference of the hollow microneedle.

8. The method of claim 5 wherein the another of the at least one nonconductive pattern are on either side of the recess.

9. The method of claim 1 wherein forming at least one electrically nonconductive pattern on a portion of the seed layer further comprises:
dispersing non-conductive nano particles into a conductive material to form a solution.

10. A hollow plated microneedle comprising:
a base;
a hollow tip;
an opening laterally offset from the tip; and
at least one sensory area.

11. The microneedle of claim 10 wherein the at least one sensory area comprises a channel.

12. The microneedle of claim 10 wherein the at least one sensory area comprises two sensory areas in the base wherein each of the two sensory areas are on either side of the hollow tip.

13. The microneedle of claim 10 wherein the at least one sensory area comprises a plurality of sensory channels wherein at least one of the plurality of sensory channels is on an exterior side of the base and at least one of the plurality of sensory channels is on an interior side of the base.

14. The microneedle of claim 10 wherein the at least one sensory area is on an interior sidewall of the hollow tip.

15. The microneedle of claim 10 further comprising an inner surface and an outer surface whereby the inner surface includes a sensory area.

16. A method of performing a biosensory function comprising:
utilizing a hollow microneedle to perform the biosensory function.

17. The method of claim 16 wherein the hollow microneedle includes at least one sensory area and utilizing the hollow microneedle to perform the biosensory function:
functionalizing the at least one sensory area to become reactive to a predetermined agent.

18. The method of claim 16 wherein the at least one sensory area comprises a channel.

19. The method of claim 16 wherein the hollow microneedle includes a tip and utilizing the hollow microneedle to perform the biosensory function further comprises:
coupling sensors to the microneedle; and
using the sensors to detect a flow or presence of an a desired agent.

20. The method of claim 17 wherein functionalizing the at least one sensory area further comprises:
functionalizing the at least one sensory area to function in a hydrophobic fashion.

21. The method of claim 17 wherein functionalizing the at least one sensory area further comprises:
functionalizing the at least one sensory area to function in a hydrophilic fashion.

22. A hollow plated microneedle comprising:
a base;
a hollow tip;
an opening laterally offset from the tip; and
micro/nano porous channels.
